(19) United States
(12) Patent Application Publication
(10) Pub. No.: US 2009/0004110 A1
(43) Pub. Date: Jan. 1, 2009

(54) Multi-functional gelatin particle and its use

(75) Inventor: Hiroyuki Yonekawa, Bethlehem, PA (US)

Correspondence Address: Thomas Spinelli;Scully Scott, Murph & Presser, PC., 400 Garden City Plaza, Suite 300, Garden City, NY, 11530, US

(73) Assignees: Olympus America Inc. Scientific Equipment Group, Center Valley, PA (US); Olympus Corporation Diagnostic systems group., Tokyo (JP)

(21) Appl. No.: 11/823,500

(22) Filed: Jun. 29, 2007

(51) Int. Cl. C12Q 1/00; A61K 49/00; A61P 43/00

(52) U.S. Cl. 424/9.1; 435/4

(57) ABSTRACT

This invention is a continuing submission of multi-functional gelatin particle and its use, provisional patent application 60/880,734 filed at 01/17/2007. And this invention is related to former patent applications of 11/698,966, 11/788,829, 11/799,487. Multi-functional gelatin particle has been used for the variety of biological assays and applications. In this patent application, we extend to use multi-functional gelatin particles as for the in vivo & in vitro shuttle assay.

We modified particle preparation steps and then, we can add extra functions in inside and outside of gelatin particles. Such multi-modified and functioned gelatin particles worked for the encapsulation of cells and serve subcutaneous injection too. Such Multi-functional gelatin particles can work as molecular indicator, target molecule isolator, and local area stimulator for the living cells also. Coming from such multi-functionalities, multi-functional gelatin particles will play a carrier of drug delivery into the cells as well as recipient animals. We can utilize every function as molecular attachment, magnetic attraction, fluorescence activation, chemicals release and transformation of its microenvironment altogether. Further we applied simple optical layout, so called tilt angled illumination for the optimized observation tool of gelatin particles when such multi-functional gelatin particle are embedded in the thick medium. Evoked two focal planes by tilt angled illumination, we can observe both side of multi-functional gelatin particle in deeper location of matrix.

### BACKGROUND OF THE INVENTION

The gelatin particle has been used in many biological assays. Especially surface modified gelatin particle has been used in clinical diagnostic field (ref 1). Then, we invented novel protocol for preparing triple functional micro-particles with temperature dependent melting for the molecular diagnostic field. (Ref 2). Gelatin particles are used for the encapsulation of cells or biological materials and implant into the animal body as in vivo use (ref 3).

The gelatin particles have high absorption capacity of target molecules and inert nature, which comes from their ubiquitous biological materials. In this patent application, we prepared potent gelatin particles through the multi-step preparation. Then we describe new application of gelatin particles, which work as micro shuttle vessels in vivo and in vitro seamlessly.

### SUMMARY OF THE INVENTION

We applied extra steps on the particle preparation process for obtaining drug-releasing capability; target molecule capture, cell entrapment, magnetic attraction, fluorescent or colored stained and multiplex handling capability. With applying weak cross-linking process, gelatin particles can incorporate several substances in inside and be isolated efficient manner. They can work as passive drug carrier depending on their surrounding environment as well as target molecule isolation. We tried to observe these functionalities and obtain images of gelatin particles, even in thick material by the tilt angled illumination.

### Embodiment 1

### Temperature Sensitive Fluorescence Labeled Micro-Particles

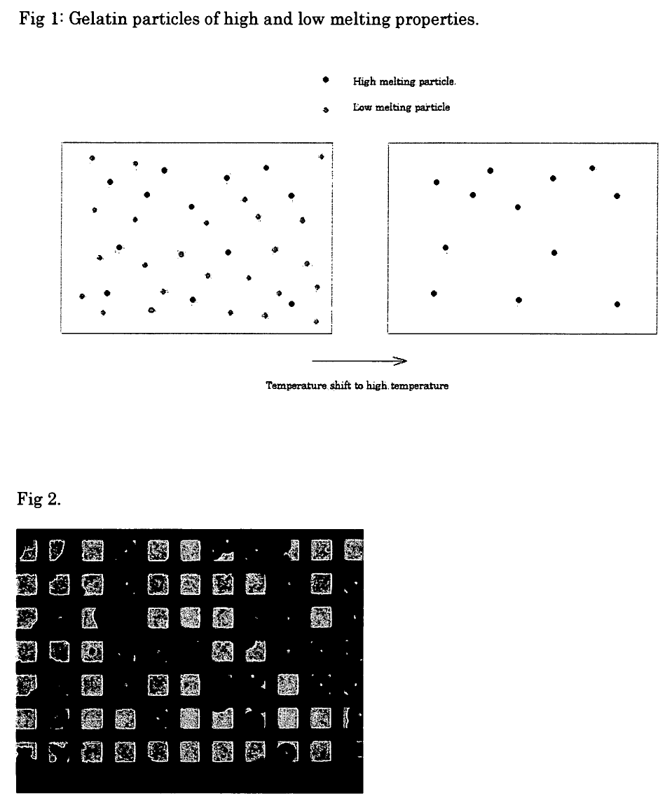

Temperature sensitive gelatin particles are mainly prepared according to the method of ref 2. Gelatin was obtained from Accurate Chemical Scientific Corp, Westbury N.Y. 11590. Arabic gum came from Senba Touka Kougyou, Japan. Acetic acids came from Heinz Pa. Phosphate buffer, Potassium hydroxide, sodium chloride and other chemicals were purchased from Sigma Aldrich, St Louis, Mo. 63178. Heated plate came from Fisher scientific. Automated mixer was built in-house. We skipped cross-linking step by aldehyde in prior art. Differently prepared gelatin particles show “melting based separation characteristics” at determined temperature as seen in (FIG. 1). We can prepare stable particles as well as melting particles according to their composition. Stable particles exist even in high temperature. In this case, low melting particles can release drugs according to the tiny increase of temperature. These gelatin particles can work as temperature or optical sensitive chemical carrier as well as cell encapsulation carrier.

### Application 1

### Immunological Absorption and Activation of Target Cells in Vitro with Temperature Shift

Gelatin particles can attach many binding molecules on their surface as immuno-globulins, lectins or ligands (ref 1). Fluorescence labeled gelatin particles are easily identified by fluorescence microscope with their target molecules. After binding to their target molecules or cells, we can apply strong excitation light or infrared illumination in order to increase the temperature of gelatin particles. It causes melting of low melting point gelatin particles. Then gelatin particles release chemical substances into their surrounding. Usually such pinpoint stimulation of small area was done by mechanical procedures as microinjection by single action manner. But by optical activation technique of gelatin particles, mass activation process can be achieved.

### Application 2

### Intravital Screening in Vivo Assay

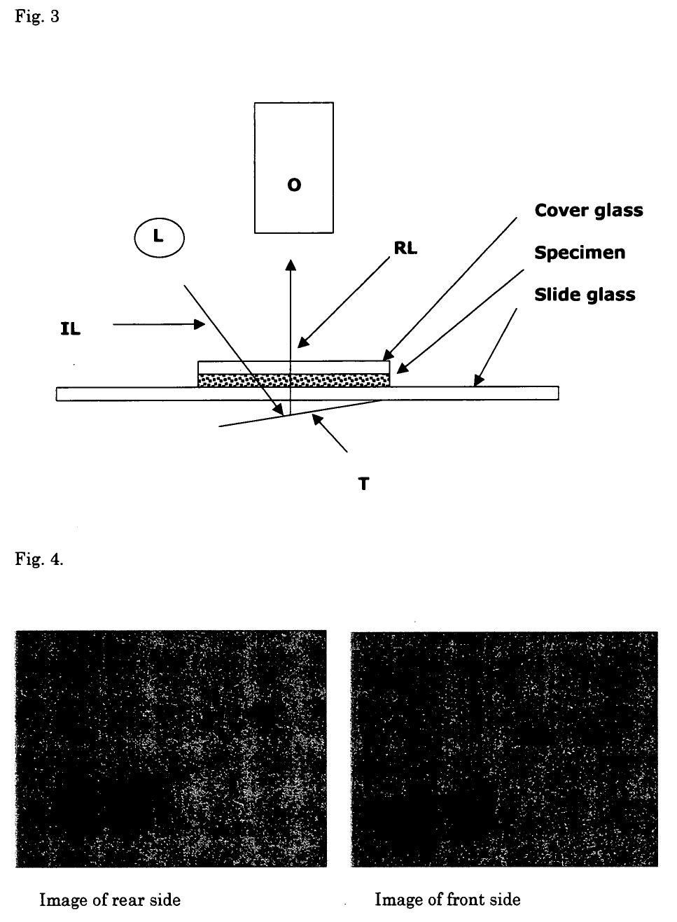

Multi-functional gelatin particle can be applied into the intravital multiplex screening system as (ref 4). Multi-functional gelatin particles are embedded into the array with target cells (FIG. 3), and then implanted into the subcutaneous of animals. Target response of embedded cells can be observed by suitable measurement device as IV100: intravital microscope or OV100: whole mouse observation system, Olympus America Inc. According to this application, we can obtain multiple cellular responses at once in native condition. Then target gelatin particles can be isolated after the measurement. In this case, multi-functional gelatin particles work as shuttle vesicles between in vitro and in vivo experiments.

### EXAMPLE 2

### Apply Tilt Angled Illumination.

Multi-functional gelatin particles can work in the thick matrix. Usually such thick matrix is not suitable for the conventional microscope, especially samples are embedded in the translucent materials.

The principle of tilt-angled illumination is illustrated in FIG. 3. The incident light (IL) that is emitted from lamp (L) goes through the slide glass or first filter (F), then thick specimen (SP) and slide glass (S). The reflecting light (RL) passes from surface of tilter (T: tilting mirror or water immense second cover glass) into the specimen (SP) again. Then that reflected light passed the object (O) in thick specimen (SP) and goes to the objective lens.

The performance of tilt-angled illumination is confirmed by simple experiment by the combination of gelatin particles with human epithelial cells and magnetic particles in thick gelling material (FIG. 4). This image is taken by MIC-D microscope (Olympus America Inc). Gelatin particles have diameter around 100 micrometers. Matrix has the depth of 2-3 mm of translucent agar. Inside of thick material can be observed, even though with the relatively translucent material as commercial gelling materials.

### EXAMPLE 3

### Fluorescent Observation by Tilt Angled Illumination

By the nature of interference filter, only shorter wavelength of light can pass the interference filter in case of angled incident light. Then, tilt angled illumination with single interference filter can work as simple fluorescence measurement optics. Using MIC-D microscope with single interference filter, fluorescence material can be observed as FIG. 6. The specimen is fluorescence labeled gelatin particle in 3 mm silicon rubber.

Detailed Description of FIG. 1.

Effect of temperature shift on gelatin particles for the high and low temperature. Low melting particles disappear in the solution in high temperature, but high melting particles still exist as particle forms. Then we can isolate the soluble fraction, which is released from the low-melting particles. Or particle fraction can be separated as ordinal magnetic particle separation manner.

Detailed Description of FIG. 2:

Arrayed matrix was supplied from Wavesense, Calif. USA. Each matrix has 100-micrometer square. Particles are embedded in each cubicle in the arrayed matrix. Further details are described in the patent application 11/698,966.

FIG. 3: A Schematic Optical Layout of Tilt Angled Illumination.

The incident light (IL), which is emitted from the light source (L) go through the cover glass, specimen and slide glass. Then that incident light (IL) reflects by tilting mirror (T). The reflected light (RL) passes through the slide glass, specimen and cover glass. The reflected light (RL) goes into the objective lens (O). The image of specimen is taken by objective lens (O) through the reflected light (RL) as well as incident light (IL) together. We can observe both side of target specimen in FIG. 4.

FIG. 4: Magnetic Particles (Black Color) Embedded in the Agar Matrix.

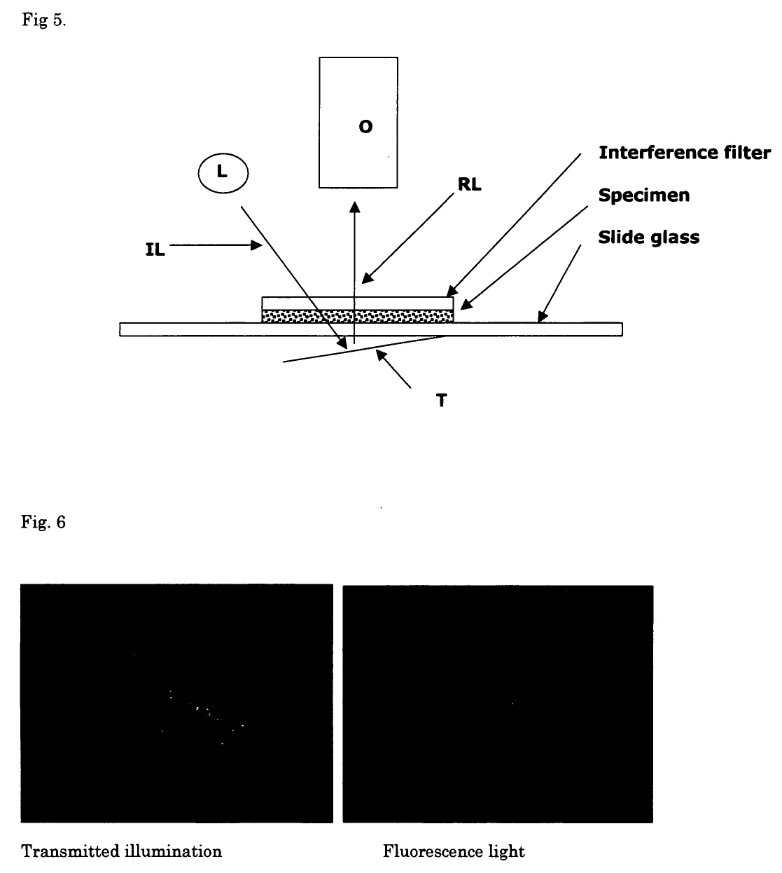

FIG. 5: A schematic optical layout of tilt angled illumination. The illumination light (IL), which is emitted from the light source (L) go through the interference filter, specimen and slide glass. Then the illumination light (IL) is reflected by tilting mirror (T). The reflected light (RL) passes the slide glass, specimen and interference filter. The reflected light (RL) goes into the objective lens (O). The image of specimen is taken by objective lens (O). By the nature of interference filter, the wavelength of transmitted light (RL) is longer than reflected light (IL). Then we can observe fluorescence image of target specimen as FIG. 6.

FIG. 6: Fluorescence Observation of Specimen by Tilt Angled Illumination

6a:Straight light for bright field image

6b:Tilt angled fluorescence image

### CROSS REFERENCE

Ref 1: JP 2716227

Ref 2: JPA 2000-275227

Ref 3: U.S. Pat. No. 5,912,005

Ref 4: U.S. patent application Ser. No. 11/698,966

Ref 5: Handbook of Fluorescent Probes and Research products. by Richard P.Haugland, Molecular Probes, Inc. 29851 Willow Creek Rd, Eugene, Oreg., USA

1. Artificially prepared multi-functional gelatin particles, which have different properties on their surface as well as inside of particles.

2. Artificially prepared multi-functional gelatin particles of claim 1, wherein said gelatin particles have fluorescence, phosphorescence or calorimetric labeling.

3. Artificially prepared multi-functional gelatin particles of claim 1, wherein said gelatin particles have magnetic property.

4. Artificially prepared multi-functional gelatin particles of claim 1, wherein said gelatin particles have cell encapsulation.

5. Artificially prepared multi-functional gelatin particles of claim 1, wherein said gelatin particles have targeted molecule attachment.

6. Artificially prepared multi-functional gelatin particles of claim 1, wherein said gelatin particles have molecular releasing depend on their environment.

7. Biological assays by said multifunctional gelatin particles in-vitro.

8. Biological assays by said multifunctional gelatin particles in-vivo.

9. Observation of said gelatin particles by tilt angled illumination.
